(19) United States
(12) Patent Application Publication
(10) Pub. No.: US 2017/0079907 A1
(43) Pub. Date: Mar. 23, 2017

(54) Sublingual Epinephrine Spray

(71) Applicant: Insys Development Company, Inc., Chandler, AZ (US)

(72) Inventors: Thrimoorthy Potta, Phoenix, AZ (US); Craig Bastian, Gilbert, AZ (US); Ningxin Yan, Chandler, AZ (US); Venkat Goskonda, Phoenix, AZ (US)

(21) Appl. No.: 15/264,686

(22) Filed: Sep. 14, 2016

Related U.S. Application Data
(60) Provisional application No. 62/220,320, filed on Sep. 18, 2015.

(51) Int. Cl. A61K 9/00; A61K 47/38; A61K 47/02; A61K 47/18; A61K 9/08; A61K 31/137

(52) U.S. Cl. CPC A61K 9/006; A61K 9/08; A61K 31/137; A61K 47/02; A61K 47/183; A61K 47/38

(57) ABSTRACT

The present invention is directed to sublingual epinephrine spray formulations. The present invention is further directed to methods of treating anaphylaxis by administering sublingual epinephrine spray formulations to subjects in need of such treatments.

### FIELD OF THE INVENTION

The present invention is directed to sublingual epinephrine spray formulations. The present invention is further directed to methods of treating anaphylaxis by administering sublingual epinephrine spray formulations to subjects in need of such treatments.

### BACKGROUND OF THE INVENTION

Epinephrine (i.e. adrenaline) is a catecholamine with the following chemical structure:

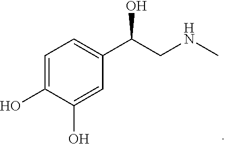

Epinephrine stimulates the alpha- and beta-adrenergic receptors of the sympathetic nervous system. Epinephrine binds to these adrenergic receptors leading to relief of many life-threatening symptoms of anaphylaxis including: relaxation of the smooth muscle in the bronchi of the lungs opening up the constricted airways; constriction of the blood vessels leading to decreased swelling of the tongue and throat and increased blood pressure; and finally increased heart rate preventing or reversing cardiovascular collapse.

Epinephrine hydrochloride is commercially available as an injection (Adrenalin® a trademark of and available from Par Sterile Products, LLC) and an auto-injector (EpiPen® a trademark of and available from Mylan, Inc. and Auvi-Q® a trademark of and available from Sanofi Corporation). Epinephrine hydrochloride was previously available as a nasal spray (Adrenalin®) and an aerosol spray (Primatene® Mist trademark of Armstrong Pharmaceuticals, Inc.). Racepinephrine is commercially available as a 2.25% oral inhalation solution for use in nebulizers (52® is available from Nephron Pharmaceuticals, Inc.). Epinephrine differs from racepinephrine in that epinephrine consists of only the L-isomer and racepinephrine is a 50/50 mixture of both the L- and D-isomers.

U.S. Pat. No. 8,628,805 is directed to a stable liquid adrenaline/bisulfite composition wherein the molar ratio of adrenaline to bisulfite is 1.31-2.20:1. U.S. Patent Application Publication No. 2012/0322884 A1 is directed to epinephrine nanoparticles, which can be incorporated into sublingual tablets. U.S. Patent Application Publication No. 2007/0202163 A1 is directed to epinephrine tablets for sublingual administration, which contain between 12% and 48% epinephrine. World Intellectual Property Organization (“W.I.P.O.”) Publication No. 2014/127018 A1 is directed to a stable aqueous epinephrine formulation that requires cyclodextrin. W.I.P.O. Publication No. 2014/057365 A1 is directed to an injectable epinephrine formulation.

“Sublingual” means “under the tongue” and refers to administration of a substance via the mouth in such a way that the substance is rapidly absorbed via the blood vessels under the tongue. A sublingual formulation is desirable because it bypasses hepatic first pass metabolic processes which provide better bioavailability, rapid onset of action, and higher patient compliance. Dysphagia (difficulty in swallowing) is common among all ages of people and more common in geriatric patients. In terms of permeability, the sublingual area of oral cavity is more permeable than the buccal area. Sublingual drug administration is applied in the fields of cardiovascular drugs, analgesics, steroids, enzymes and barbiturates.

While there are various epinephrine formulations currently available, there remains a need in the art for a quick-onset sublingual spray formulation.

### SUMMARY OF THE INVENTION

In one aspect, the present invention is directed to sublingual epinephrine spray formulations comprising epinephrine base, or a salt thereof, preferably from about 0.5% to about 10.0% w/w epinephrine base, or a salt thereof.

In another aspect, the present invention is directed to sublingual epinephrine spray formulations comprising epinephrine base, or a salt thereof, wherein the formulation is free of a propellant.

In another aspect, the present invention is directed to sublingual epinephrine spray formulations comprising epinephrine base, or a salt thereof, and an acid.

In another aspect, the present invention is directed to sublingual epinephrine spray formulations comprising epinephrine base, or a salt thereof, and a solvent.

In another aspect, the present invention is directed to sublingual epinephrine spray formulations comprising epinephrine base, or a salt thereof, and a stabilizer.

In another aspect, the present invention is directed to sublingual epinephrine spray formulations comprising from about 1% to about 3% w/w epinephrine base, or a salt thereof, from about 1% to about 50% w/w 0.5 N hydrochloric acid and from about 63% to about 99% w/w water.

In another aspect, the present invention is directed to sublingual epinephrine spray formulations comprising from about 1% to about 3% w/w epinephrine base, or a salt thereof, from about 1% to about 50% w/w 0.5 N hydrochloric acid, from about 1% to about 50% w/w ethanol, from about 0.5% to about 10% w/w propylene glycol, and from about 1% to about 20% w/w water.

In another aspect, the present invention is directed to sublingual epinephrine spray formulations comprising from about 0.5% to about 10% w/w epinephrine base, or a salt thereof, from about 1% to about 50% w/w 0.5 N hydrochloric acid, from about 5% to about 55% w/w alcohol, from about 0.01% to about 10% w/w menthol, from about 5% to about 50% propylene glycol and from about 1% to about 20% w/w water.

In another aspect, the present invention is directed to a method of treating anaphylaxis comprising administering to a subject in need thereof a sublingual epinephrine spray formulation of the present invention.

### DETAILED DESCRIPTION OF THE INVENTION

### Definitions

Applicants unexpectedly discovered sublingual epinephrine spray formulations that have improved bioavailability, a more rapid onset of action, and improved storage stability.

As used herein, “epinephrine” refers to the base or an ester, derivative, or prodrug thereof.

As used herein, “free of propellant” refers to a formulation that is not administered using compressed gas.

As used herein, all numerical values relating to amounts, weights, and the like, that are defined as “about” each particular value is plus or minus 10%. For example, the phrase “about 10% w/w” is to be understood as “9% w/w to 11% w/w.” Therefore, amounts within 10% of the claimed value are encompassed by the scope of the claims.

As used herein “% w/w” and “percent w/w” refer to the percent weight of the total formulation.

As used herein the term “effective amount” refers to the amount necessary to treat a patient in need thereof.

As used herein the term “treat”, “treating” or “treatment” refers to ameliorating or inhibiting symptoms of anaphylaxis.

As used herein the term “subject” refers, but is not limited to, a person that is experiencing anaphylaxis.

As used herein the term “anaphylaxis” refers to an allergic reaction involving multiple organ systems in a subject upon contact with an allergen rather or not that allergen is identifiable.

As used herein the term “allergen” refers to any chemical capable of causing an immune system response in a subject including, but not limited to, chemicals found in drugs, food, plants, insect bites, and insect stings.

As used herein the term “pharmaceutically acceptable” refers to ingredients that are not biologically or otherwise undesirable in a sublingual dosage form.

In one embodiment, the present invention is directed to sublingual epinephrine spray formulations comprising epinephrine base, or a salt thereof.

Preferred epinephrine salts include citrate, hydrochloride, halide, sulfate, tartrate, phosphate, acetate, malate, maleate, succinate, ascorbate, carbonate, mesylate and lactate. One of skill in the art could use other pharmaceutically acceptable epinephrine salts in the formulations of the present invention. In a preferred embodiment, the formulations contain the pharmaceutically acceptable salt equivalent to from about 0.1% to about 30% w/w epinephrine. In a more preferred embodiment, the formulation contains the pharmaceutically acceptable salt equivalent to from about 0.5% to about 20% w/w of epinephrine. Other most preferred embodiments include formulations which contain the pharmaceutically acceptable salt equivalent to from about 0.5% w/w to about 10% w/w, from about 1% to about 3% w/w, or about 1% w/w or about 3% w/w of epinephrine

In another embodiment, the present invention is directed to sublingual epinephrine spray formulations comprising epinephrine base, or a salt thereof, wherein the formulation is free of a propellant.

In another embodiment, the present invention is directed to sublingual epinephrine spray formulations comprising epinephrine base, or a salt thereof, and one or more excipients selected from acids, solvents, stabilizers, permeation enhancers, viscosity modifiers, sweeteners, sweetness enhancers, pH modifiers and flavoring agents.

In a preferred embodiment, the formulations of the present invention contain from about 1% to about 50% w/w of an acid, more preferably from about 1% to about 40% w/w, and even more preferably about 10.66% w/w, 32.6% w/w or 35.20% w/w. Acids suitable for use in the present invention include, but are not limited to, hydrochloric acid, malic acid, tartaric acid, citric acid, succinic acid and combinations thereof. In a preferred embodiment the acid is hydrochloric acid or malic acid, even more preferably 0.1N to 12N hydrochloric acid and most preferably 0.5N hydrochloric acid.

In a preferred embodiment, the formulations of the present invention contain more than about 10% w/w, more than about 20% w/w, more than about 30% w/w, more than about 40% w/w, more than about 50% w/w, more than about 60% w/w, more than 70% w/w, more than about 80% w/w, more than about 90% w/w of a solvent, preferably from about 50% to about 99.9% w/w, and more preferably from about 63% to about 99% w/w. Solvents suitable for use in the present invention include, but are not limited to, water, ethanol, glycerin, propylene glycol, polyethylene glycol 400 and combinations thereof. In a preferred embodiment the solvent is water. In another preferred embodiment the solvent is a combination of ethanol, propylene glycol and water.

In a preferred embodiment, the formulations of the present invention contain from about 0.001% to about 5% w/w of a stabilizer, preferably from about 0.11% to about 1.1% w/w or about 0.02% to about 2% w/w, and even more preferably from about 0.055% to about 0.5% w/w. Stabilizers suitable for use in the present invention include, but are not limited to, butylated hydroxyanisole (BHA), butylated hydroxytoluene (BHT), ascorbic acid, methionine, sodium ascorbate, sodium thiosulfate, sodium bisulfite, sodium metabisulfite, ascorbyl palmitate, thioglycerol, alpha tocopherol (vitamin E), cysteine hydrochloride, citric acid, ethylenediaminetetraacetic acid (“EDTA”), sodium citrate, propyl gallate, 8-hydroxyquinoline, boric acid, histidine and combinations thereof. In a preferred embodiment the stabilizer is selected from sodium metabisulfite, sodium bisulfite, EDTA, 8-hydroxyquinoline and combinations thereof. In an even more preferred embodiment the stabilizer is selected from sodium metabisulfite, sodium bisulfite, EDTA and combinations thereof.

In some embodiments, the formulations of the present invention contain from about 0.001% w/w to about 10% w/w of a permeation enhancer, more preferably from about 0.05% w/w to about 5% w/w, and even more preferably from about 0.5% to about 1% w/w.

Permeation enhancers suitable for use in the present invention include, but are not limited to, oleic acid, polysorbate 80, menthol, EDTA, sodium edentate, cetylpyridinium chloride, sodium lauryl sulfate, citric acid, sodium desoxycholate, sodium deoxyglycolate, glyceryl oleate, L-lysine and combinations thereof.

Viscosity modifiers suitable for the present invention include, but are not limited to, polyvinylpyrrolidone, carboxymethyl cellulose, hydroxypropylmethyl cellulose (“HPMC”), methyl cellulose, hydroxyethyl cellulose, glycerin, polyvinyl alcohol and combinations thereof. In a preferred embodiment the viscosity modifier is HPMC.

Sweeteners suitable for the present invention include, but are not limited to, sucralose, sucrose, aspartame, saccharin, dextrose, mannitol, glycerin, xylitol and combinations thereof.

In some embodiments, the formulations of the present invention contain from about 0.001% to about 1% of a sweetness enhancer. Sweetness enhancers suitable for the present invention include, but are not limited to, the ammonium salt forms of crude and refined Glycyrrhizic Acid. Magnasweet® products (available from Mafco Worldwide Corporation, Magnasweet is a registered trademark of Mafco Worldwide Corporation) use the ammonium salt forms of crude and refined Glycyrrhizic Acid. Glycyrrhizic Acid is also available as a pure derivative in the sodium and potassium salt forms.

In a preferred embodiment, the formulations of the present invention are at a pH from about 2.0 to about 5.0. pH modifiers suitable for the present invention include, but are not limited to, hydrochloric acid, citric acid, fumaric acid, lactic acid, sodium hydroxide, sodium citrate, sodium bicarbonate, sodium carbonate, ammonium carbonate and combinations thereof.

Preservatives suitable for the present invention include, but are not limited to, butyl paraben, methyl paraben, ethyl paraben, propyl paraben, sodium benzoate, chlorobutanol, benzoic acid and combinations thereof.

Flavoring agents suitable for the present invention include, but are not limited to, peppermint oil, menthol, spearmint oil, citrus oil, cinnamon oil, strawberry flavor, cherry flavor, raspberry flavor, orange oil and a combination thereof.

In preferred embodiment, the present invention is directed to sublingual epinephrine spray formulations comprising from about 1% to about 3% w/w epinephrine base, or a salt thereof, from about 1% to about 50% w/w 0.5 N hydrochloric acid and from about 63% to about 99% w/w water.

In another preferred embodiment, the present invention is directed to sublingual epinephrine spray formulations comprising from about 1% to about 3% w/w epinephrine base, or a salt thereof, from about 1% to about 50% w/w 0.5 N hydrochloric acid, from about 63% to about 99% w/w water and a stabilizer selected from EDTA at a concentration from about 0.01% to about 0.1% w/w, sodium metabisulfite at a concentration from about 0.001% to about 2% w/w, sodium bisulfite at a concentration from about 0.001% to about 2% w/w and a combination thereof.

In another preferred embodiment, the present invention is directed to sublingual epinephrine spray formulations comprising from about 1% to about 3% w/w epinephrine base, or a salt thereof, from about 1% to about 50% w/w 0.5 N hydrochloric acid, from about 63% to about 99% w/w water, a stabilizer selected from EDTA at a concentration from about 0.01% to about 0.1% w/w, sodium metabisulfite at a concentration from about 0.001% to about 2% w/w, sodium bisulfite at a concentration from about 0.001% to about 2% w/w and a combination thereof, and 8-hydroxyquinoline at a concentration from about 0.01% to about 0.05% w/w or sodium chloride at a concentration from about 0.1% to about 1% w/w.

In a more preferred embodiment, the present invention is directed to a sublingual epinephrine spray formulation comprising about 1.0% w/w epinephrine base, about 10.66% w/w 0.5 N hydrochloric acid, about 0.05% w/w EDTA, about 0.025% w/w sodium metabisulfite, about 0.025% w/w sodium bisulfite and about 88.24% w/w water.

In a most preferred embodiment, the present invention is directed to a sublingual epinephrine spray formulation comprising about 3.0% w/w epinephrine base, about 32.6% w/w 0.5 N hydrochloric acid, about 0.5% w/w chlorobutanol, about 0.5% w/w sucralose, about 0.05% w/w EDTA, about 0.2% w/w sodium bisulfite and about 63.15% w/w water.

In another embodiment, the present invention is directed to sublingual epinephrine spray formulations comprising from about 1% to about 3.0% w/w epinephrine base, or a salt thereof, from about 1% to about 50% w/w 0.5 N hydrochloric acid, from about 1% to about 50% w/w ethanol, from about 0.5% to about 10% w/w propylene glycol, and from about 1% to about 20% w/w water.

In another embodiment, the present invention is directed to sublingual epinephrine spray formulations comprising from about 1% to about 3.0% w/w epinephrine base, or a salt thereof, from about 1% to about 50% w/w 0.5 N hydrochloric acid, from about 1% to about 50% w/w ethanol, from about 0.5% to about 5% w/w propylene glycol, from about 0.1% to about 1% w/w sucralose, from about 1% to about 20% w/w water and a stabilizer selected from EDTA at a concentration from about 0.01% to about 0.1% w/w and sodium bisulfite at a concentration from about 0.001% to about 2% w/w.

In another preferred embodiment, the present invention is directed to a sublingual epinephrine spray formulation comprising about 3.0% w/w epinephrine base, about 32.6% w/w 0.5 N hydrochloric acid, about 50% w/w ethanol, about 5% w/w propylene glycol, about 5% w/w menthol, about 0.5% w/w sucralose, about 0.05% w/w EDTA, about 0.2% w/w sodium bisulfite and about 4.15% w/w water.

In another embodiment, the formulations of the present invention are capable of producing a droplet size distribution at 3 cm wherein the mean DV(10) is from about 15 to about 18 microns during administration.

In another embodiment, the formulations of the present invention are capable of producing a droplet size distribution at 3 cm wherein the mean DV(50) is from about 30 to about 34 microns during administration.

In another embodiment, the formulations of the present invention are capable of producing a droplet size distribution at 3 cm wherein the mean DV(90) is from about 120 to about 230 microns during administration.

In another embodiment, the formulations of the present invention are capable of producing a spray span ((Dv90−Dv10)/Dv50) at 3 cm of from about 3 to about 7.

In another embodiment, the formulations of the present invention are capable of producing a droplet size distribution at 6 cm wherein the mean DV(10) is from about 22 to about 25 microns during administration.

In another embodiment, the formulations of the present invention are capable of producing a droplet size distribution at 6 cm wherein the mean DV(50) is from about 36 to about 41 microns during administration.

In another embodiment, the formulations of the present invention are capable of producing a droplet size distribution at 6 cm wherein the mean DV(90) is from about 59 to about 231 microns during administration.

In another embodiment, the formulations of the present invention are capable of producing a spray span ((Dv90−Dv10)/Dv50) at 6 cm of from about 1 to about 6.

In another embodiment, the formulations of the present invention are capable of producing a spray pattern at 3 cm wherein the Dmin is from about 18 to about 23 millimeters during administration.

In another embodiment, the formulations of the present invention are capable of producing a spray pattern at 3 cm wherein the Dmax is from about 29 to about 33 millimeters during administration.

In another embodiment, the formulations of the present invention are capable of producing a spray pattern at 3 cm wherein the ovality ratio is from about 1.4 to about 1.7 during administration.

In another embodiment, the formulations of the present invention are capable of producing a spray pattern at 6 cm wherein the Dmin is from about 26 to about 33 millimeters during administration.

In another embodiment, the formulations of the present invention are capable of producing a spray pattern at 6 cm wherein the Dmax is from about 47 to about 52 millimeters during administration.

In another embodiment, the formulations of the present invention are capable of producing a spray pattern at 6 cm wherein the ovality ratio is from about 1.6 to about 1.9 during administration.

In another embodiment, the formulations of the present invention are capable of producing a plume geometry at 3 cm wherein the angle is from about 49° to about 64°.

In another embodiment, the formulations of the present invention are capable of producing a plume geometry at 3 cm wherein the width is from about 27 to about 38 millimeters.

In another embodiment, the formulations of the present invention are capable of producing a plume geometry at 3 cm wherein the angle is from about 37° to about 44°.

In another embodiment, the formulations of the present invention are capable of producing a plume geometry at 3 cm wherein the width is from about 37 to about 44 millimeters.

The disclosed embodiments are simply exemplary embodiments of the inventive concepts disclosed herein and should not be considered as limiting, unless the claims expressly state otherwise.

The following examples are intended to illustrate the present invention and to teach one of ordinary skill in the art how to use the formulations of the invention. They are not intended to be limiting in any way.

### Examples

### Example 1

An epinephrine spray was prepared as follows using the components and amounts listed in Table 1 below. All of the solvents were purged with nitrogen prior to use. Excipients including 0.5 N hydrochloric acid (“HCl”), malic acid, ethanol and propylene glycol, EDTA, sodium chloride, sodium bisulfate, sodium metabisulfite, and 8-hydroxyquinoline were dissolved in water while stirring at room temperature. Epinephrine base was then added to the excipient solution. Finally, sodium hydroxide (“NaOH”) was used to adjust final pH.

TABLE 1
Epinephrine Formulations

| | % w/w | | | | | | | | | | |
|---|---|---|---|---|---|---|---|---|---|---|---|
| | #1 | #2 | #3 | #4 | #5 | #6 | #7 | #8 | #9 | #10 | #11 |
| Epinephrine base | 1.0 | 1.0 | 1.0 | 1.0 | 1.0 | 7.5 | 1 | 3.0 | 3.0 | 3.0 | 3.0 |
| HCl (0.5N) | 10.66 | 10.66 | 10.66 | 10.66 | 10.66 | — | 11.35 | — | 32.6 | 32.6 | 32.6 |
| EDTA | 0.05 | 0.05 | 0.05 | 0.05 | 0.05 | — | — | — | 0.05 | 0.05 | 0.05 |
| Sodium | 0.01 | 0.015 | 0.02 | 0.025 | 0.025 | 0.25 | 0.025 | 0.04 | — | — | — |
| metabisulfite | | | | | | | | | | | |
| sodium bisulfite | 0.01 | 0.015 | 0.02 | 0.025 | 0.025 | — | 0.025 | | 0.05 | 0.2 | 0.2 |
| Propylene Glycol | — | — | — | — | — | — | — | — | — | — | 5 |
| Menthol | — | — | — | — | — | — | — | — | — | — | 5 |
| Chlorobutanol | — | — | — | — | — | — | — | — | — | 0.5 | — |
| Sucralose | — | — | — | — | — | — | — | — | — | 0.5 | 0.5 |
| 8- | — | — | — | — | 0.02 | — | — | — | — | — | — |
| Hydroxyquinoline | | | | | | | | | | | |
| Ethanol | — | — | — | — | — | 15 | | — | — | — | 50 |
| Malic Acid | — | — | — | — | — | 2.0 | — | 1.65 | — | — | — |
| Water | 88.27 | 88.26 | 88.25 | 88.24 | 88.22 | 79.75 | 87.6 | 95.31 | 64.3 | 63.15 | 4.15 |
| | 100 | 100 | 100 | 100 | 100 | 100 | 100 | 100 | 100 | 100 | 100 |
| pH adjusted with | pH | pH | pH | pH | pH | pH | pH | pH | pH | pH | pH |
| NaOH | 4.5 | 4.5 | 4.5 | 3.7 | 4.5 | 4.5 | 4.5 | 4.5 | 4.5 | 4.5 | 4.5 |

### Example 2

The formulations listed in Table 1 were subjected to stability at 40° C.±2° C./75%±5% relative humidity and 25° C.±2° C./60%±5% relative humidity. The stability of the formulations were analyzed at specified time points by evaluating their potency (assay value) and impurity levels. Assay and impurities were detected using high-performance liquid chromatography with an ultraviolet detector. The assay was performed at 280 nm and indicated as a % of initial concentration. For all impurities, analysis was performed at 210 nm and 280 nm and expressed as a % area. Amounts of particular impurities are listed in Tables 2 to 14 as a percentage of area of each formulation along with amount of total impurities. Relative retention time (“RRT”) is given for each impurity. “ND” indicates that the impurity was not detected.

TABLE 2
Stability Data for Sublingual Epinephrine Spray Formulation #1 stored
at 40° C. ± 2° C./75% ± 5% Relative Humidity

| 40° C. Formulation #1 | RRT | 0 Week | 2 Weeks | 4 Weeks |
|---|---|---|---|---|
| Appearance | | Clear | Clear | Clear |
| Assay (% of initial conc.) | | 100.00 | 97.94 | 95.80 |
| % Racemization | | 0.60 | 0.93 | 1.54 |
| pH | | 4.50 | 3.52 | 3.29 |
| % Impurity F | 0.19 | 0.13 | 1.07 | 1.51 |
| % Synephrine | 1.26 | ND | ND | ND |
| % Epinephrone | 1.36 | ND | ND | ND |
| % Methoxy | 1.88 | 0.07 | 0.07 | 0.07 |
| % Unknown Impurity | 0.21 | ND | 0.10 | 0.15 |
| | 0.23 | ND | ND | 0.02 |
| | 0.27 | ND | ND | ND |
| | 0.74 | ND | ND | ND |
| | 0.88 | ND | ND | ND |
| | 2.48 | ND | ND | ND |
| | 3.11 | ND | 0.01 | 0.03 |
| | 3.26 | ND | 0.01 | 0.02 |
| % Total Impurities | | 0.20 | 1.26 | 1.80 |

TABLE 3
Stability Data for Sublingual Epinephrine Spray Formulation #2
stored at 40° C. ± 2° C./75% ± 5% Relative Humidity

| 40° C. Formulation #2 | RRT | 0 Week | 2 Weeks | 4 Weeks |
|---|---|---|---|---|
| Appearance | | Clear | Clear | Clear |
| Assay (% of initial conc.) | | 100.00 | 96.02 | 97.32 |
| % Racemization | | 0.60 | 0.91 | 1.77 |
| pH | | 4.50 | 3.39 | 3.14 |
| % Impurity F | 0.19 | 0.14 | 1.27 | 1.91 |
| % Synephrine | 1.26 | ND | ND | ND |
| % Epinephrone | 1.38 | ND | ND | 0.01 |
| % Methoxy | 1.88 | 0.07 | 0.07 | 0.07 |
| % Unknown Impurity | 0.21 | ND | 0.10 | 0.11 |
| | 0.23 | ND | ND | 0.01 |
| | 0.27 | ND | ND | ND |
| | 0.74 | ND | ND | ND |
| | 0.88 | ND | ND | ND |
| | 2.48 | ND | ND | ND |
| | 3.11 | ND | ND | ND |
| | 3.26 | ND | ND | ND |
| % Total Impurities | | 0.21 | 1.44 | 2.11 |

TABLE 4
Stability Data for Sublingual Epinephrine Spray Formulation #3 stored at 40° C. ±
2° C./75% ± 5% Relative Humidity

| 40° C. Formulation #3 | RRT | 0 Week | 2 Weeks | 4 Weeks | 8 Weeks | 3 Months |
|---|---|---|---|---|---|---|
| Appearance | | Clear | Clear | Clear | Clear | Clear |
| Assay (% of initial conc.) | | 100.00 | 98.22 | 96.87 | 94.56 | 93.27 |
| % Racemization | | 0.60 | 1.01 | 1.48 | 3.67 | 4.23 |
| pH | | 4.50 | 3.37 | 3.14 | 3.01 | — |
| % Impurity F | 0.19 | 0.13 | 1.27 | 2.19 | 3.05 | 3.18 |
| % Synephrine | 1.26 | ND | ND | ND | ND | ND |
| % Epinephrone | 1.36 | ND | ND | 0.01 | 0.01 | 0.03 |
| % Methoxy | 1.88 | 0.07 | 0.07 | 0.07 | 0.07 | 0.07 |
| % Unknown Impurity | 0.21 | ND | 0.08 | 0.07 | 0.08 | 0.24 |
| | 0.23 | ND | ND | ND | ND | ND |
| | 0.27 | ND | ND | ND | ND | ND |
| | 0.74 | ND | ND | ND | ND | ND |
| | 0.88 | ND | ND | ND | ND | ND |
| | 2.48 | ND | ND | ND | ND | ND |
| | 3.11 | ND | ND | 0.02 | 0.02 | 0.04 |
| | 3.26 | ND | ND | 0.02 | 0.02 | 0.04 |
| % Total Impurities | | 0.20 | 1.42 | 2.38 | 3.25 | 3.60 |

TABLE 5
Stability Data for Sublingual Epinephrine Spray Formulation #4 stored at 40° C. ±
2° C./75% ± 5% Relative Humidity

| 40° C. | | | | | | | | | |
|---|---|---|---|---|---|---|---|---|---|
| Formulation | | 0 | 1 | 2 | 4 | 6 | 8 | 3 | 4 |
| #4 | RRT | Week | Week | Weeks | Weeks | Weeks | Weeks | Months | Months |
| Appearance | | Clear | Clear | Clear | Clear | Clear | Clear | Clear | Clear |
| Assay (% of | | 100.0 | 100.7 | 98.54 | 98.35 | 96.02 | 94.44 | 92.60 | 89.34 |
| initial conc.) | | | | | | | | | |
| % | | 0.60 | 0.68 | — | — | 3.41 | 3.46 | 5.75 | 9.20 |
| Racemization | | | | | | | | | |
| % Impurity F | 0.19 | 0.15 | 0.78 | 1.29 | 2.33 | 3.16 | 3.83 | 4.36 | 4.65 |
| % Synephrine | 1.26 | ND | ND | ND | ND | ND | ND | ND | ND |
| % | 1.36 | ND | ND | ND | 0.01 | 0.01 | 0.02 | 0.01 | 0.02 |
| Epinephrone | | | | | | | | | |
| % Methoxy | 1.88 | 0.08 | 0.08 | 0.08 | 0.08 | 0.08 | 0.08 | 0.08 | 0.09 |
| % Unknown | 0.21 | ND | ND | 0.05 | 0.08 | 0.07 | 0.09 | 0.22 | 0.23 |
| Impurity | 0.23 | ND | ND | ND | ND | ND | ND | ND | ND |
| | 0.26 | ND | ND | ND | ND | ND | ND | ND | ND |
| | 0.88 | ND | ND | ND | ND | ND | ND | ND | ND |
| | 2.31 | ND | ND | ND | ND | ND | ND | ND | ND |
| | 2.58 | ND | ND | ND | ND | ND | ND | ND | ND |
| | 3.11 | ND | ND | ND | 0.02 | 0.02 | 0.02 | 0.02 | 0.05 |
| | 3.26 | ND | ND | ND | 0.02 | 0.02 | 0.02 | 0.02 | 0.03 |
| % Total | | 0.23 | 0.86 | 1.42 | 2.54 | 3.36 | 4.06 | 4.71 | 5.07 |
| Impurities | | | | | | | | | |

TABLE 6
Stability Data for Sublingual Epinephrine Spray Formulation #5 stored at 40° C. ±
2° C./75% ± 5% Relative Humidity

| 40° C. Formulation #5 | RRT | 0 Week | 2 Weeks | 4 Weeks | 8 Weeks | 3 Months |
|---|---|---|---|---|---|---|
| Appearance | | Clear | Clear | Clear | Clear | Clear |
| Assay (% of initial conc.) | | 100.00 | 98.36 | 96.88 | 95.35 | 93.34 |
| % Racemization | | 0.60 | 0.85 | 1.08 | 2.63 | 4.23 |
| pH | | 4.50 | 3.64 | 3.37 | 3.20 | — |
| % Impurity F | 0.19 | 0.15 | 1.48 | 2.50 | 3.50 | 3.75 |
| % Synephrine | 1.26 | ND | ND | ND | ND | ND |
| % Epinephrone | 1.36 | ND | ND | 0.01 | 0.01 | 0.03 |
| % Methoxy | 1.88 | 0.07 | 0.07 | 0.07 | 0.07 | 0.07 |
| % Unknown Impurity | 0.21 | ND | 0.10 | 0.11 | 0.15 | 0.35 |
| | 0.23 | ND | ND | ND | ND | ND |
| | 0.27 | ND | ND | ND | ND | ND |
| | 0.74 | ND | ND | ND | ND | ND |
| | 0.88 | ND | ND | ND | ND | 0.02 |
| | 2.48 | ND | ND | ND | ND | ND |
| | 3.11 | ND | ND | 0.02 | 0.02 | 0.01 |
| | 3.26 | ND | ND | 0.01 | 0.02 | 0.01 |
| % Total Impurities | | 0.22 | 1.65 | 2.72 | 3.77 | 4.24 |

TABLE 7
Stability Data for Sublingual Epinephrine Spray Formulation #6
stored at 40° C. ± 2° C./75% ± 5% Relative Humidity

| 40° C. Formulation #6 | RRT | 0 Week | 2 Weeks | 1 Month |
|---|---|---|---|---|
| Appearance | | Clear | Clear | Clear |
| Assay (% of initial conc.) | | 100.00 | 97.75 | 95.95 |
| % Impurity F | 0.19 | 0.15 | 2.38 | 4.04 |
| % Synephrine | 1.26 | ND | ND | ND |
| % Epinephrone | 1.36 | ND | 0.01 | 0.01 |
| % Methoxy | 1.88 | 0.04 | 0.04 | 0.04 |
| % Unknown Impurity | 0.21 | ND | 0.05 | 0.08 |
| | 0.23 | ND | ND | ND |
| | 0.26 | ND | ND | ND |
| | 0.88 | ND | ND | ND |
| | 2.31 | ND | ND | ND |
| | 2.58 | ND | ND | ND |
| | 3.11 | ND | ND | ND |
| | 3.26 | ND | ND | ND |
| % Total Impurities | | 0.23 | 2.55 | 4.25 |

TABLE 8
Stability Data for Sublingual Epinephrine Spray Formulation #7
stored at 40° C. ± 2° C./75% ± 5% Relative Humidity

| 40° C. Formulation #7 | RRT | 0 Week | 2 Weeks | 4 Weeks |
|---|---|---|---|---|
| Appearance | | Clear | Clear | Clear |
| % Impurity F | 0.19 | 0.32 | 1.85 | 2.75 |
| % Synephrine | 1.26 | ND | ND | ND |
| % Epinephrone | 1.36 | ND | ND | ND |
| % Methoxy | 1.88 | 0.07 | 0.07 | 0.07 |
| % Unknown Impurity | 0.21 | 0.17 | 0.36 | 0.44 |
| | 0.23 | ND | ND | ND |
| | 0.26 | ND | ND | ND |
| | 0.88 | ND | ND | ND |
| | 2.31 | ND | ND | ND |
| | 2.58 | ND | ND | ND |
| | 3.11 | ND | ND | ND |
| | 3.26 | ND | ND | ND |
| % Total Impurities | | 0.56 | 2.28 | 3.26 |

TABLE 9
Stability Data for Sublingual Epinephrine Spray Formulation #8
stored at 40° C. ± 2° C./75% ± 5% Relative Humidity

| 40° C. Formulation #8 | RRT | 0 Week | 2 Weeks |
|---|---|---|---|
| Appearance | | Clear | Light Brown |
| Assay (% of initial conc.) | | 100.00 | 95.55 |
| % Racemization | | 0.63 | 0.62 |
| % Impurity F | 0.19 | 0.22 | 1.56 |
| % Synephrine | 1.26 | ND | ND |
| % Epinephrone | 1.36 | ND | ND |
| % Methoxy | 1.88 | 0.07 | 0.07 |
| % Unknown Impurity | 0.21 | ND | 0.12 |
| | 0.23 | ND | ND |
| | 0.26 | ND | 0.03 |
| | 0.88 | ND | 0.03 |
| | 2.31 | ND | ND |
| | 2.58 | ND | ND |
| | 3.11 | ND | 0.02 |
| | 3.26 | ND | 0.02 |
| % Total Impurities | | 0.27 | 4.68 |

TABLE 10
Stability Data for Sublingual Epinephrine Spray Formulation #9
stored at 40° C. ± 2° C./75% ± 5% Relative Humidity

| 40° C. Formulation #9 | RRT | 0 Week | 1 Week | 4 Weeks |
|---|---|---|---|---|
| Appearance | | Clear | Clear | Clear |
| Assay (% of initial conc.) | | 100.00 | 100.76 | 99.73 |
| % Racemization | | 0.60 | 0.68 | — |
| % Impurity F | 0.19 | 0.15 | 0.84 | 1.86 |
| % Synephrine | 1.26 | ND | ND | ND |
| % Epinephrone | 1.36 | ND | ND | ND |
| % Methoxy | 1.88 | 0.07 | 0.07 | 0.07 |
| % Unknown Impurity | 0.21 | ND | ND | 0.06 |
| | 0.23 | ND | ND | ND |
| | 0.26 | ND | ND | ND |
| | 0.88 | ND | ND | ND |
| | 2.31 | ND | ND | ND |
| | 2.58 | ND | ND | ND |
| | 3.11 | ND | 0.04 | 0.09 |
| | 3.26 | ND | 0.04 | 0.08 |
| % Total Impurities | | 0.22 | 1.00 | 2.17 |

TABLE 11
Stability Data for Sublingual Epinephrine Spray Formulation #10
stored at 40° C. ± 2° C./75% ± 5% Relative Humidity

| 40° C. Formulation #10 | RRT | 0 Week | 1 Week |
|---|---|---|---|
| Appearance | | Clear | Clear |
| Assay (% of initial conc.) | | 100.00 | 100.76 |
| % Racemization | | 0.60 | 0.68 |
| % Impurity F | 0.19 | 0.12 | 2.89 |
| % Synephrine | 1.26 | ND | ND |
| % Epinephrone | 1.36 | ND | ND |
| % Methoxy | 1.88 | 0.07 | 0.07 |
| % Unknown Impurity | 0.21 | ND | 0.02 |
| | 0.23 | ND | ND |
| | 0.26 | ND | ND |
| | 0.88 | ND | ND |
| | 2.31 | ND | ND |
| | 2.58 | ND | ND |
| | 3.11 | ND | 0.04 |
| | 3.26 | ND | 0.04 |
| % Total Impurities | | 0.22 | 3.74 |

TABLE 12
Stability Data for Sublingual Epinephrine Spray Formulation #11 stored
at 40° C. ± 2° C./75% ± 5% Relative Humidity

| 40° C. Formulation #11 | RRT | 0 Week | 1 Week |
|---|---|---|---|
| Appearance | | Clear | Clear |
| Assay (% of initial conc.) | | 100.00 | 100.76 |
| % Racemization | | 0.60 | 0.68 |
| % Impurity F | 0.19 | 0.12 | 0.90 |
| % Synephrine | 1.26 | ND | ND |
| % Epinephrone | 1.36 | ND | ND |
| % Methoxy | 1.88 | 0.07 | 0.07 |
| % Unknown Impurity | 0.21 | ND | 0.03 |
| | 0.23 | ND | ND |
| | 0.26 | ND | ND |
| | 0.88 | ND | ND |
| | 2.31 | ND | ND |
| | 2.58 | ND | ND |
| | 3.11 | ND | 0.04 |
| | 3.26 | ND | 0.04 |
| % Total Impurities | | 0.22 | 1.00 |

TABLE 13
Stability Data for Sublingual Epinephrine Spray Formulation #1 stored
at 25° C. ± 2° C./60% ± 5% Relative Humidity

| 25° C. Formulation #1 | RRT | 0 Week | 1 Month | 3 Months |
|---|---|---|---|---|
| Appearance | | Clear | Clear | Clear |
| Assay (% of initial | | 100.00 | 97.91 | 97.32 |
| conc.) | | | | |
| % Racemization | | 0.60 | 0.76 | 0.93 |
| % Impurity F | 0.19 | 0.13 | 0.53 | 1.04 |
| % Synephrine | 1.26 | ND | ND | ND |
| % Epinephrone | 1.36 | ND | ND | ND |
| % Methoxy | 1.88 | 0.07 | 0.07 | 0.07 |
| % Unknown Impurity | 0.21 | ND | 0.05 | 0.19 |
| | 0.23 | ND | ND | ND |
| | 0.26 | ND | ND | ND |
| | 0.88 | ND | ND | ND |
| | 2.58 | ND | ND | ND |
| | 3.11 | ND | ND | 0.04 |
| | 3.26 | ND | ND | 0.03 |
| % Total Impurities | | 0.20 | 0.65 | 1.37 |

TABLE 14
Stability Data for Sublingual Epinephrine Spray Formulation #2 stored
at 25° C. ± 2° C./60% ± 5% Relative Humidity

| 25° C. Formulation #2 | RRT | 0 Week | 1 Month | 3 Months |
|---|---|---|---|---|
| Appearance | | Clear | Clear | Clear |
| Assay (% of initial | | 100.00 | 97.56 | 96.88 |
| conc.) | | | | |
| % Racemization | | 0.60 | 0.78 | 1.01 |
| % Impurity F | 0.19 | 0.14 | 0.55 | 1.25 |
| % Synephrine | 1.26 | ND | ND | ND |
| % Epinephrone | 1.36 | ND | ND | ND |
| % Methoxy | 1.88 | 0.07 | 0.07 | 0.07 |
| % Unknown Impurity | 0.21 | ND | 0.08 | 0.18 |
| | 0.23 | ND | ND | ND |
| | 0.26 | ND | ND | ND |
| | 0.88 | ND | ND | ND |
| | 2.58 | ND | ND | ND |
| | 3.11 | ND | ND | 0.02 |
| | 3.26 | ND | ND | 0.03 |
| % Total Impurities | | 0.21 | 0.70 | 1.55 |

TABLE 15
Stability Data for Sublingual Epinephrine Spray Formulation #3 stored
at 25° C. ± 2° C./60% ± 5% Relative Humidity

| 25° C. Formulation #3 | RRT | 0 Week | 1 Month | 3 Months |
|---|---|---|---|---|
| Appearance | | Clear | Clear | Clear |
| Assay (% of initial | | 100.00 | 99.32 | 98.16 |
| conc.) | | | | |
| % Racemization | | 0.60 | 0.75 | 0.84 |
| % Impurity F | 0.19 | 0.13 | 0.52 | 1.29 |
| % Synephrine | 1.26 | ND | ND | ND |
| % Epinephrone | 1.36 | ND | ND | ND |
| % Methoxy | 1.88 | 0.07 | 0.07 | 0.07 |
| % Unknown Impurity | 0.21 | ND | 0.03 | 0.09 |
| | 0.23 | ND | ND | ND |
| | 0.26 | ND | ND | ND |
| | 0.88 | ND | ND | ND |
| | 2.58 | ND | ND | ND |
| | 3.11 | ND | ND | 0.02 |
| | 3.26 | ND | ND | 0.03 |
| % Total Impurities | | 0.20 | 0.62 | 1.50 |

TABLE 16
Stability Data for Sublingual Epinephrine Spray Formulation #4 stored at 25° C. ±
2° C./60% ± 5% Relative Humidity

| | | | 3 | 4 | 8 | 3 | 6 |
|---|---|---|---|---|---|---|---|
| 25° C. Formulation #4 | RRT | 0 Week | Weeks | Weeks | Weeks | Months | Months |
| Appearance | | Clear | Clear | Clear | Clear | Clear | Clear |
| Assay (% of initial | | 100.00 | 100.76 | 99.79 | 99.31 | 99.09 | 98.69 |
| conc.) | | | | | | | |
| % Racemization | | 0.60 | 0.68 | — | 0.91 | 0.97 | — |
| % Impurity F | 0.19 | 0.15 | 0.41 | 0.50 | 1.01 | 1.46 | 2.39 |
| % Synephrine | 1.26 | ND | ND | ND | ND | ND | ND |
| % Epinephrone | 1.36 | ND | ND | ND | ND | 0.01 | 0.01 |
| % Methoxy | 1.88 | 0.08 | 0.08 | 0.08 | 0.08 | 0.08 | 0.08 |
| % Unknown Impunty | 0.21 | ND | 0.05 | 0.06 | 0.06 | 0.11 | 0.10 |
| | 0.23 | ND | ND | ND | ND | ND | ND |
| | 0.26 | ND | ND | ND | ND | ND | ND |
| | 0.88 | ND | ND | ND | ND | ND | ND |
| | 2.31 | ND | ND | ND | ND | ND | ND |
| | 3.02 | ND | ND | ND | ND | ND | 0.12 |
| | 3.11 | ND | ND | ND | ND | ND | 0.12 |
| | 3.26 | ND | ND | ND | ND | ND | 0.10 |
| % Total Impurities | | 0.23 | 0.54 | 0.64 | 1.15 | 1.66 | 2.92 |

TABLE 17
Stability Data for Sublingual Epinephrine Spray Formulation #5 stored
at 25° C. ± 2° C./60% ± 5% Relative Humidity

| 25° C. Formulation #5 | RRT | 0 Week | 1 Month | 3 Months |
|---|---|---|---|---|
| Appearance | | Clear | Clear | Clear |
| Assay (% of initial | | 100.00 | 98.40 | 97.29 |
| conc.) | | | | |
| % Racemization | | 0.60 | 0.77 | 0.84 |
| % Impurity F | 0.19 | 0.15 | 0.67 | 1.41 |
| % Synephrine | 1.26 | ND | ND | ND |
| % Epinephrone | 1.36 | ND | ND | ND |
| % Methoxy | 1.88 | 0.07 | 0.07 | 0.07 |
| % Unknown Impurity | 0.21 | ND | 0.05 | 0.18 |
| | 0.23 | ND | ND | ND |
| | 0.26 | ND | ND | ND |
| | 0.88 | ND | ND | ND |
| | 2.58 | ND | ND | ND |
| | 3.11 | ND | ND | 0.01 |
| | 3.26 | ND | ND | 0.01 |
| % Total Impurities | | 0.22 | 0.79 | 1.68 |

TABLE 18
Stability Data for Sublingual Epinephrine Spray Formulation #6 stored
at 25° C. ± 2° C./60% ± 5% Relative Humidity

| 25° C. | | | | | |
|---|---|---|---|---|---|
| Formulation #6 | RRT | 0 Week | 2 Weeks | 1 Month | 6 Months |
| Appearance | | Clear | Clear | Clear | Clear |
| Assay (% of | | 100.00 | 100.25 | 99.75 | 94.80 |
| initial conc.) | | | | | |
| % Impurity F | 0.19 | 0.15 | 0.45 | 0.76 | 2.93 |
| % Synephrine | 1.26 | ND | ND | ND | ND |
| % Epinephrone | 1.36 | ND | 0.01 | 0.02 | 0.04 |
| % Methoxy | 1.88 | 0.04 | 0.04 | 0.04 | 0.04 |
| % Unknown | 0.21 | ND | ND | 0.04 | 0.30 |
| Impurity | 0.23 | ND | ND | ND | ND |
| | 0.26 | ND | ND | ND | 0.13 |
| | 0.88 | ND | ND | ND | 0.15 |
| | 2.31 | ND | ND | ND | ND |
| | 2.58 | ND | ND | ND | 0.10 |
| | 3.11 | ND | ND | ND | 0.24 |
| | 3.26 | ND | ND | ND | 0.17 |
| % Total | | 0.18 | 0.53 | 0.89 | 4.10 |
| Impurities | | | | | |

TABLE 19
Stability Data for Sublingual Epinephrine Spray Formulation #8 stored
at 25° C. ± 2° C./60% ± 5% Relative Humidity

| 25° C. Formulation #8 | RRT | 0 Week | 1 Month |
|---|---|---|---|
| Appearance | | Clear | Clear |
| Assay (% of initial | | 100.00 | 99.21 |
| conc.) | | | |
| % Racemization | | 0.63 | 0.66 |
| % Impurity F | 0.19 | 0.22 | 0.64 |
| % Synephrine | 1.26 | ND | ND |
| % Epinephrone | 1.36 | ND | ND |
| % Methoxy | 1.88 | 0.07 | 0.07 |
| % Unknown Impurity | 0.21 | ND | 0.06 |
| | 0.23 | ND | ND |
| | 0.26 | ND | ND |
| | 0.88 | ND | ND |
| | 2.31 | ND | ND |
| | 2.58 | ND | ND |
| | 3.11 | ND | 0.02 |
| | 3.26 | ND | 0.02 |
| % Total Impurities | | 0.29 | 0.81 |

TABLE 20
Stability Data for Sublingual Epinephrine Spray Formulation #9 stored
at 25° C. ± 2° C./60% ± 5% Relative Humidity

| 25° C. Formulation #9 | RRT | 0 Week | 1 Month | 6 Months |
|---|---|---|---|---|
| Appearance | | Clear | Clear | Clear |
| Assay (% of initial | | 100.00 | 99.64 | 98.88 |
| conc.) | | | | |
| % Impurity F | 0.19 | 0.15 | 0.52 | 1.71 |
| % Synephrine | 1.26 | ND | ND | ND |
| % Epinephrone | 1.36 | ND | ND | ND |
| % Methoxy | 1.88 | 0.08 | 0.07 | 0.07 |
| % Unknown Impurity | 0.21 | ND | 0.05 | 0.07 |
| % Unknown Impurity | 0.23 | ND | ND | ND |
| % Total Impurities | 0.26 | ND | ND | ND |
| | 0.88 | ND | ND | ND |
| | 2.31 | ND | ND | ND |
| | 2.58 | ND | ND | ND |
| | 3.11 | ND | 0.03 | 0.09 |
| | 3.26 | ND | 0.02 | 0.08 |
| % Total Impurities | | 0.23 | 0.70 | 2.02 |

Formulations #1-#5 had less than 3% total impurities after 4 Weeks (1 Month) at 40° C.±2° C./75%±5% Relative Humidity and less than 1% total impurities after 4 Weeks (1 Month) at 25° C.±2° C./60%±5% relative humidity. Of Formulations #6-#8, only Formulation #8 was analyzed at 4 Weeks or later at 40° C. where 4.68% total impurities were found. Formulation #9 exhibited total impurities of 2.17% at 4 weeks 40° C. Formulation #11 exhibited a total impurities of 1% at one week 40° C. The superior and surprising stability characteristics of the formulations of the present invention will allow the formulations to be effective when used by patients.

When compared with formulation 4, formulation 7 showed a faster generation of impurities and was not stable for more than a month at 40° C. Formulation 4 was stable for 4 months when stored at 40° C. which indicates that EDTA increases the stability of epinephrine formulations.

### Example 3

Formulation #4 was further tested for stability during freeze-thaw cycling. Specifically, Formulation #4 was run through 3 cycles of −20° C. for 48 hours and then 25° C. for 48 hours, where the physical appearance of the formulation was recorded. The formulation remained clear and colorless throughout the entire freeze-thaw cycling indicating a stable formulation throughout.

### Example 4

In order to determine the spray profile of Formulation #4, it was subjected to standardized droplet testing. A challenge of creating a sublingual epinephrine spray formulation is that it must be capable of producing spray droplets that are over 10 microns in diameter. Spray droplets of 10 microns or smaller could be inhaled into the lungs.

Droplet analysis was conducted using standard laser analysis procedures known by those of skill in the art. Droplet size distribution (Dv₁₀, Dv₅₀, Dv₉₀, and Span) was tested at two distances, 3 cm and 6 cm. Dv₁₀ refers to the droplet size at which 10% of the volume is smaller; Dv₅₀ refers to the median droplet size; Dv₉₀ refers to droplet size for which 90% of the total volume is smaller; Span refers to distribution span (Dv90−Dv10)/Dv50. %<10 μm refers to the percentage of the total volume that is made up of droplets less than 10 μm in diameter. Spray pattern, specifically Dmin, Dmax, and ovality ratio were tested at two distances, 3 cm and 6 cm. Dmin refers to the shortest diameter of the spray pattern in mm, Dmax refers to the widest diameter of the spray pattern in mm, and ovality ratio refers to the ratio of Dmax to Dmin. The spay pattern is measured by shining a laser sheet perpendicular to the spray at a specific distance from the orifice. The ovality ratio is useful as it provides information regarding the shape and density of the spray pump plume.

The results of these tests can be seen below in Tables 17 to 22.

TABLE 21
Droplet size distribution of Sublingual Epinephrine
Spray Formulation at 3 cm

| Droplet Size | | | | | |
|---|---|---|---|---|---|
| Distribution 3 cm | DV₁₀ (μm) | DV₅₀ (μm) | DV₉₀ (μm) | % <10 μm | Span |
| Min | 15.8 | 30.84 | 124 | 0.05 | 3.145 |
| Max | 17.23 | 33.96 | 228.7 | 0.89 | 6.283 |
| Mean | 16.34 | 32.88 | 177.9 | 0.473 | 4.93 |

TABLE 22
Droplet size distribution of Sublingual Epinephrine Spray
Formulation at 6 cm

| Droplet Size | | | | | |
|---|---|---|---|---|---|
| Distribution 6 cm | DV₁₀ (μm) | DV₅₀ (μm) | DV₉₀ (μm) | % <10 μm | Span |
| Min | 22.72 | 36.07 | 59.52 | 0 | 1.017 |
| Max | 24.05 | 40.35 | 230.9 | 0 | 5.127 |
| Mean | 23.2 | 38.48 | 121.5 | 0 | 2.49 |

TABLE 23
Spray pattern of Sublingual Epinephrine Spray Formulation at 3 cm

| | Dmin | Dmax | Ovality |
|---|---|---|---|
| Spray Pattern 3 cm | (mm) | (mm) | ratio |
| Min | 18.9 | 29.8 | 1.415 |
| Max | 22.2 | 32.1 | 1.696 |
| Mean | 20.4 | 31.1 | 1.53 |

TABLE 24
Spray pattern of Sublingual Epinephrine Spray Formulation at 6 cm

| | Dmin | Dmax | Ovality |
|---|---|---|---|
| Spray Pattern 6 cm | (mm) | (mm) | ratio |
| Min | 26.5 | 47 | 1.68 |
| Max | 32.2 | 54.2 | 1.852 |
| Mean | 29.1 | 51.4 | 1.768 |

TABLE 25
Plume geometry of Sublingual Epinephrine Spray Formulation at 3 cm

| | | Width |
|---|---|---|
| Plume Geometry 3 cm | Angle (°) | (mm) |
| Min | 49.2 | 27.6 |
| Max | 63.4 | 37.8 |
| Mean | 55.4 | 32.2 |

TABLE 26
Plume geometry of Sublingual Epinephrine Spray Formulation at 6 cm

| | | Width |
|---|---|---|
| Plume Geometry 6 cm | Angle (°) | (mm) |
| Min | 37.7 | 37.7 |
| Max | 43.5 | 43.5 |
| Mean | 40.7 | 40.7 |

Applicant found during testing that formulations of the present invention yielded desirable droplet sizes for sublingual administration. The testing also revealed that the formulation dose remains consistent when administered with a spray pump.

What we claim is:

1. A sublingual epinephrine spray formulation comprising epinephrine base or a salt thereof.

2. The formulation of claim 1 wherein the epinephrine base or salt thereof is at a concentration from about 0.5% to about 10.0% w/w, wherein w/w indicates weight by weight of the total formulation.

3. The formulation of claim 1 wherein the formulation is free of a propellant.

4. The formulation of claim 1 further comprising a solvent selected from the group consisting of water, ethanol, glycerin, propylene glycol, polyethylene glycol 400 and a combination thereof.

5. The formulation of claim 4 wherein the solvent is water.

6. The formulation of claim 5 wherein the solvent is a combination of ethanol, propylene glycol and water.

7. The formulation of claim 1 further comprising an acid selected from hydrochloric acid, malic acid, tartaric acid, citric acid, succinic acid and a combination thereof.

8. The formulation of claim 6 wherein the acid is hydrochloric acid.

9. The formulation of claim 1 further comprising a stabilizer selected from butylated hydroxyanisole (BHA), butylated hydroxytoluene (BHT), ascorbic acid, methionine, sodium ascorbate, sodium thiosulfate, sodium bisulfite, sodium metabisulfite, ascorbyl palmitate, thioglycerol, alpha tocopherol (vitamin E), cysteine hydrochloride, citric acid, ethylenediaminetetraacetic acid (EDTA), sodium citrate, propyl gallate, 8-hydroxyquinolone, boric acid, histidine and a combination thereof.

10. The formulation of claim 8 wherein the stabilizer is selected from sodium bisulfite, sodium metabisulfite, EDTA and a combination thereof.

11. The formulation of claim 1 further comprising a permeation enhancer selected from the group consisting of oleic acid, polysorbate 80, menthol, ethylenediaminetetraacetic acid (EDTA), sodium edentate, cetylpyridinium chloride, sodium lauryl sulfate, citric acid, sodium desoxycholate, sodium deoxyglycolate, glyceryl oleate, L-lysine and a combination thereof.

12. The formulation of claim 1 further comprising a viscosity modifier selected from the group consisting of polyvinylpyrrolidone, carboxymethyl cellulose, hydroxypropylmethyl cellulose, methyl cellulose, hydroxyethyl cellulose, glycerin, polyvinyl alcohol and a combination thereof.

13. The formulation of claim 12 wherein the viscosity modifier is hydroxypropylmethyl cellulose.

14. The formulation of claim 1 further comprising a sweetener selected from the group consisting of sucrose, sucralose, aspartame, saccharin, dextrose, mannitol, glycerin, xylitol and a combination thereof.

15. The formulation of claim 14 further comprising a sweetness enhancer.

16. The formulation of claim 15 wherein the sweetness enhancer is glycyrrhizic acid ammonium salt.

17. The formulation of claim 1 further comprising a pH modifier selected from the group consisting of hydrochloric acid, citric acid, fumaric acid, lactic acid, sodium hydroxide, sodium citrate, sodium bicarbonate, sodium carbonate, ammonium carbonate and a combination thereof.

18. The formulation of claim 1 further comprising a preservative selected from the group consisting of butyl paraben, methyl paraben, ethyl paraben, propyl paraben, sodium benzoate, chlorobutanol, benzoic acid and a combination thereof.

19. The formulation of claim 1 further comprising a flavoring agent selected from the group consisting of peppermint oil, menthol, spearmint oil, citrus oil, cinnamon oil, strawberry flavor, cherry flavor, raspberry flavor, orange oil and a combination thereof.

20. A sublingual epinephrine spray formulation comprising from about 1% to about 3% w/w epinephrine base or a salt thereof, from about 1% to about 50% w/w 0.5 N hydrochloric acid and from about 63% to about 99% w/w water, wherein w/w indicates weight by weight of the total formulation.

21. The formulation of claim 20 further comprising a stabilizer selected from ethylenediaminetetraacetic acid (EDTA) at a concentration from about 0.01% to about 0.1% w/w, sodium metabisulfite at a concentration from about 0.001% to about 2% w/w, sodium bisulfite at a concentration from about 0.001% to about 2% w/w and a combination thereof.

22. The formulation of claim 21 further comprising 8-hydroxyquinoline at a concentration from about 0.01% to about 0.05% w/w or sodium chloride at a concentration from about 0.1% to about 1% w/w.

23. The formulation of claim 20 wherein the pH is from about 2.0 to about 5.0.

24. The formulation of claim 21 wherein the epinephrine base, or salt thereof, is at a concentration of about 1.0% w/w, 0.5 N hydrochloric acid is at a concentration of about 10.66% w/w, the water is at a concentration of about 88.24% w/w, the ethylenediaminetetraacetic acid (EDTA) is at a concentration of about 0.05% w/w, the sodium metabisulfite is at a concentration of about 0.025% w/w and the sodium bisulfite is at a concentration of about 0.025% w/w.

25. The formulation of claim 20 further comprising about 0.05% w/w ethylenediaminetetraacetic acid (EDTA), about 0.2% w/w sodium bisulfite, about 0.5% w/w chlorobutanol and about 0.5% w/w sucralose, wherein the epinephrine base or salt thereof is at a concentration of about 3.0% w/w, 0.5 N hydrochloric acid is at a concentration of about 32.6% w/w and the water is at a concentration of about 63.15% w/w water.

26. A sublingual epinephrine spray formulation comprising from about 1% to about 3.0% w/w epinephrine base, or a salt thereof, from about 1% to about 50% w/w 0.5 N hydrochloric acid, from about 1% to about 50% w/w ethanol, from about 0.5% to about 10% w/w propylene glycol, and from about 1% to about 20% w/w water.

27. The formulation of claim 26 further comprising a stabilizer selected from ethylenediaminetetraacetic acid (EDTA) at a concentration from about 0.01% to about 0.1% w/w, sodium bisulfite at a concentration from about 0.001% to about 2% w/w

28. The formulation of claim 26 wherein the pH is from about 2.0 to about 5.0.

29. The formulation of claim 27 further comprising about 5% w/w menthol, about 0.5% w/w sucralose, wherein the epinephrine base or salt thereof is at a concentration of about 3.0% w/w, the 0.5 N hydrochloric acid is at a concentration of about 32.6% w/w, the ethanol is at a concentration of about 50% w/w, the propylene glycol is at a concentration of about 5% w/w, the water is at a concentration of about 4.15% w/w, the ethylenediaminetetraacetic acid (EDTA) is at a concentration of about 0.05% w/w, the sodium bisulfate is at a concentration of about 0.2% w/w.

30. A method of treating anaphylaxis comprising administering to a subject in need thereof an effective amount of a sublingual epinephrine spray formulation of claim 1, 20 or 26.
